(19) United States
(12) Patent Application Publication
(10) Pub. No.: US 2024/0321402 A1
(43) Pub. Date: Sep. 26, 2024

(54) GENERATING MOLECULES ACCOUNTING FOR REACTION CONDITIONS AND REACTION PRODUCTS

(71) Applicant: International Business Machines Corporation, Armonk, NY (US)

(72) Inventors: AKIHIRO KISHIMOTO, Setagaya (JP); Gerhard Ingmar Meijer, Zürich (CH); Valery Weber, Zürich (CH)

(21) Appl. No.: 18/188,526

(22) Filed: Mar. 23, 2023

(51) Int. Cl. G16C 20/10; G16C 20/30; G16C 20/70

(52) U.S. Cl. CPC G16C 20/10; G16C 20/30; G16C 20/70

(57) ABSTRACT

A method for predicting a reactant of a chemical reaction which results in a reaction product, such that a boundary condition is satisfied, is disclosed. The method comprises receiving records of molecule descriptions and related characteristic property values, generating training data for a machine-learning (ML) system for predicting a predefined characteristic property value of reaction products related to a reactant, and combining, for received records of the molecule descriptions, sub-structures of molecules described by the molecule descriptions using chemical rules to generate candidate reactants. Furthermore, the method comprises predicting, using the ML system, a predefined characteristic property value related to candidate reactants, whereby the candidate reactants are separately used as input for the trained machine-learning system, where the machine-learning system has been trained using the training data, and filtering out all candidate reactants for which a condition related to the predicted predefined characteristic property value is not met.

### BACKGROUND

The present disclosure relates generally to a method for predicting a reactant of a chemical reaction.

Recently, significant progress has been made in computer-aided material discovery to identify, e.g., chemical structures having predefined chemical and/or physical properties. This way, significant improvements could be made in material sciences. Such chemical structures have already been introduced in a plurality of different fields, like drug design, drug detection, but also for finding new materials—e.g., photoresists for advanced semiconductor production processes. Thereby, current approaches for molecular generation may consider structural constraints of the generated molecules, e.g., the number of types of substructures that appear in the molecule that is generated. Typically, such methods and systems may make use of machine-learning systems.

### SUMMARY OF THE INVENTION

According embodiments of the present disclosure, a computer-implemented method for predicting a reactant of a chemical reaction which results in at least one reaction product, where, at least one reaction product satisfies at least one boundary condition may be provided. The method may comprise receiving a plurality of records of molecule descriptions, where each record comprises a codified description of a molecule and related at least one characteristic property value of the molecule. The method may further comprise generating, using the plurality of records of molecule descriptions, training data for a machine-learning system adapted for predicting a predefined characteristic property value of a set of reaction products relating to a reactant, where the reactant R and the set of reaction products relate to each other according to a chemical equation R→set of reaction products, combining, for received records of the molecule descriptions, sub-structures of molecules described by the molecule descriptions using chemical rules to generate a set of candidate reactants, predicting, using the machine-learning system in a trained form, a predefined characteristic property value relating to candidate reactants, whereby the candidate reactants are separately used as input for the trained machine-learning system, where the machine-learning system has been trained using the training data, and filtering out all candidate reactants for which a condition relating to the predicted predefined characteristic property value is not met.

According to further embodiments of the present disclosure, a system and computer program product for performing the method are provided.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 shows a flow diagram of a computer-implemented method for predicting a reactant of a chemical reaction which results in at least one reaction product where the at least one reaction product satisfies at least one boundary condition, according to embodiments.

FIG. 2 shows a block diagram of an example method and related system components, according to embodiments.

FIG. 3 depicts an example of a candidate reactant for a boundary condition requiring at least one metal atom, according to embodiments.

FIG. 4 shows a block diagram of an embodiment of the inventive reactant prediction system for predicting a reactant of a chemical reaction which results in at least one reaction product, wherein the at least one reaction product satisfies at least one boundary condition.

FIG. 5 shows an embodiment of a computing system comprising the system according to FIG. 4.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present disclosure relates generally to a method for predicting a reactant of a chemical reaction, and more specifically, to a method for predicting a reactant of a chemical reaction which results in at least one reaction product, wherein the at least one reaction product satisfies at least one boundary condition. While the present disclosure is not necessarily limited to such applications, various aspects of the disclosure may be appreciated through a discussion of various examples using this context.

Currently available molecular generation methods can only consider a molecule itself and no synthesis in which the molecule participates. Therefore, current methods take into account neither reaction conditions nor reaction products. Although the existing approaches may access libraries and other tools (e.g., EPA Toxicity Estimation Software [TEST]) to estimate, e.g., given characteristic property values of the artificially generated molecules, the boundary conditions relating to potential reaction products generated from those molecules remain unconsidered. Also, advanced molecules description methods like Policy Gradient for Forward Synthesis (PGFS) only imply reinforcement learning and reaction rules to generate the molecule structures optimized for an objective function (e.g., QED). However, even such advanced methods do not consider the properties of reaction products of the target molecules.

Hence, there remains a need to reflect predefined boundary conditions when applying molecule generation methods.

In the context of this disclosure, the following technical conventions, terms and/or expressions may be used:

The term ‘chemical reaction’ may denote a process that leads to a chemical transformation of a set of chemical substances—in particular, at least one chemical substance—to form at least one reaction product. Typically, a plurality of chemical substances—also denoted as reactants—may be transformed to reaction products.

The term ‘predicting a reactant’ may denote an activity of forecasting a chemical reactant if one or more characteristic values of one or more reaction products are used as input values for a prediction system, e.g., a trained machine-learning system.

The term ‘boundary condition’ may denote a condition under which the chemical reaction may happen. The condition may be related to process parameters, environmental parameters, and/or requirements regarding the reaction products: in general any pre-definable condition under which the chemical reaction may happen. As an example, one may define that the reaction products all have a boiling point temperature below a predefined temperature value. Or that at least one reaction product comprises a metal atom or that at least one reaction product is produced during the chemical transformation what has an acid character or would be an amino acid. It should also be noted again that the boundary condition could not really be reflected under traditional processes for predicting reactants, reaction products and some related property values. Furthermore, single boundary conditions may be combined to a collective boundary condition as the single boundary condition for the proposed concept.

The term ‘molecule description’ may denote a predefined code to unambiguously characterize a chemical substance, i.e., a molecule. Various description codes are known. Some of them may be interpreted by a computer system. One of them is denoted ‘SMILES” (Simplified Molecular-Import Line-Entry System). Here, a chemical formula may be expressed as a specification in the form of a line notation using an ASCII string. Such ASCII strings may also be converted back into the well-known two-dimensional or three-dimensional models of the related molecules. Hence, the expression ‘codified description of a molecule’ may denote in the context of this disclosure such ASCII-type strings.

The term ‘characteristic property value’ may generally denote any property value of a chemical substance or molecule. Examples may comprise the melting point temperature value, the boiling point temperature value, an absorption frequency value, a membership of a predefined chemical group, and so on.

The term ‘machine-learning system’ may denote a computational system enabled to execute operations in the field of machine-learning (ML). In contrast to classical procedural programming, ML systems may be able to learn from examples—typically denoted as ‘training data’—in order to predict a certain outcome. Typical ML models are directed to classifications or regression type of predictions. Characteristic elements of the ML system may be described by hyper-parameters, whereas characteristic values of nodes of the ML system may be described as parameters—or better—parameter values of the ML system. During the training process of the ML system a tuned set of parameter values may be generated which may be denoted as the ML model of the trained machine-learning system. Types of ML systems used for the concept proposed here may comprise kernel ridge regression (KRR), support vector regression (SVR) and a wide range of others.

The term ‘sub-structure of molecule’ may denote a radical or an intermediate radical of a chemical reaction. A sub-structure of a molecule may also denote a chemical group, like the methyl (CH₃) group, just to name one example. The sub-structure may also be only one ion or atom (e.g., a metal atom).

The term ‘candidate reactants’ may denote a group of potential reactants which may have been predicted by the trained machine-learning system. The candidate reactants would need to be able to undergo a chemical reaction in order to produce groups of reaction products with at least in parts predefined characteristic property values.

The term ‘activation energy’ may denote here, in particular, the bond dissociation energy. In the context of this document, the maximum bond dissociation energy of the bonds of a reactant may be used which may be broken according to a reaction template. Thereby, using a reaction template is a known technology.

The term ‘outlier’ may denote a data point that may differ significantly—i.e., more than a predefined value—from other related observed data points. Thereby, generating an outlier may have different reasons. One of them may be a variability in the measurement or it may be the result of an experimental error. In typical experiment sequences, outliers may be eliminated from the measured data. However, in other cases an outlier may also be an indication for novel data which should be researched in more detail.

The proposed computer-implemented method for predicting a reactant of a chemical reaction which results in at least one reaction product, wherein the at least one reaction product satisfies at least one boundary condition, may offer multiple advantages, technical effects, contributions and/or improvements:

The proposed concept may take a new approach in the field of computer-added material discovery in that reaction conditions and reaction products are taken into account. In general, at least one boundary condition for a chemical reaction of reactants to reaction products can be reflected, not only for the reactants but also for the reaction products. E.g., as boundary condition, the highest melting point of all reaction products may be predefined. As another example, the boundary condition may describe and require that at least one reaction products may comprise at least one metal atom. Many other boundary conditions may be definable.

Based on this inventive concept, new materials may become discoverable in the field of semiconductor production process chemistry, drug detection, environmental technology (“green chemistry”). The at least one boundary condition may also relate to process parameters, e.g., in order to reduce the energy consumption if predefined reaction products should be produced. The concept proposed here goes well beyond the ability of existing methods to predict models created with predefined properties. One of the reasons may lie in the fact that the concept proposed here not only looks at the reactant itself but also at reaction products of the used reactant.

Hence, the search space of molecule structures may be reduced substantially. In other words, much more relevant molecular structures may be generated in a given computing system in a given amount of time. By using reaction rules, it is possible to determine reaction products in a simple yet basic manner. Hence, the ability to control the molecular generation—whether constrained by reaction conditions or the underlying chemical process—may help to increase the chances of discovering more appropriate molecular structures.

Furthermore, the proposed concept is combinable with existing virtual lab concepts like MolGX (part of the known Generic Toolkit for Scientific Discovery, GT4SD) such that the traditional concepts may be enhanced by the novel approach. Actually, the newly proposed concept may be embedded into the existing approaches and processes. None of these conventional tools allow constraints on reaction conditions or constraints on reaction products.

In the following, additional embodiments of the inventive concept-applicable for the method as well as for the system-will be described.

According to some embodiments, the predefined characteristic property value may be the highest boiling point temperature of the at least one of the reaction product of a set of reaction products or an activation energy required to facilitate a chemical reaction according to R→set of reaction products, where R is a reactant, and where “→” is read as “reacts to form. One interesting option of the underlying machine-learning system may be that both the highest boiling point temperature of the at least one of the reaction products and the required activation energy to facilitate the chemical reaction may be predicted by the same trained ML system. On the other side, it may also be possible to predict the highest boiling point temperature and the required activation energy by separate trained ML systems. Furthermore, it may be possible to predict the named value by the same ML system but using differently trained machine-learning models.

According to some embodiments, the generating the training data may comprise determining a set of reaction products for each of the records of molecule descriptions, whereby molecules relating to the molecule descriptions are used as reactants. Thereby, one may assume that at least one reactant typically results in one set of reaction products. For this determination, known techniques may be used. This part of the prediction process may be unconditional so that existing concepts (like MolGX) may be used.

Furthermore, embodiments may also comprise determining a characteristic property value for each reaction product in each set of reaction products being determined for each of the plurality of molecules used as reactants, and augmenting each record of molecule descriptions with a predefined value of the characteristic property values of the set of the determined reaction products which is related to the corresponding record of the molecule descriptions. Thereby, the at least one characteristic value may exemplary be the boiling point temperature. In general, physical as well as chemical property values may be determined. As a basis, a library comprising such physical and/or chemical property values of known chemical substances may be used.

According to embodiments, the filtering out may also comprise determining more accurate values of the characteristic property values for each reaction product relating to the set of generated candidate reactants. Also for this, a library with the related values of the molecules may be used. Furthermore, a known traditional algorithm or machine-learning approach may be applied.

These embodiments may also comprise filtering out all the candidate reactants for which the condition relating to the more accurate values of the characteristic property values is not met.

According to some embodiments, the reactant R may comprise at least two reactants, and the reaction products comprising only one reaction product. Such a combination may, e.g., be used as a drug discovery test. Thereby, R1 may denote the drug to be discovered, R2 may be a detection molecule for the chemical reaction

R1+R2→P, such that the product P may easily be detectable.

It may also be noted that the proposed concept may also support the more general description of a chemical reaction, namely

R1+R2+ . . . +Rn→P1+P2+ . . . Pm, where Ri may describe reactants, Pi may describe reaction products and where n and m may be integer numbers. Thereby, for the generated Ri—in particular by the ML system—all of P1, P2, . . . , Pm may have a desired thermal, chemical, and/or physical property values, such as a desired solubility, a desired viscosity, a desired diffusion constant, and/or a desired mechanical property (e.g., Young's modulus), just to name a few. The desired characteristics may also be combined into one or a combined boundary condition.

According to another embodiment, the boundary condition or an environmental condition of the chemical reaction may be an exposure to a radiation source. One example of such radiation source may be an extreme ultraviolet (EUV) radiation source. This may allow to discover advanced photoresist materials for a production of semiconductor components having structure sizes of 5 nm and below and which need to be illuminated with light of specifically short wavelengths.

According to another embodiment, the at least one characteristic property value in the plurality of records of molecule descriptions may comprise an activation energy for a molecule described by the related molecule description of the record. Hence, reverse prediction may be addressed by the ML system, where not characteristic property values of the reactant itself may be predicted, but those of the reaction products of the underlying chemical transformation or chemical reaction.

According to another embodiment, the generation of the set of candidate reactants may also comprise identifying outliers in the set of candidate reactants regarding their chemical properties. This may enable a more straightforward global process for predicting reactants and avoid side arms of potential reaction options which may be characterized as “non-promising”.

According to another embodiment, the method may also comprise generating an alert signal and presenting the identified outliers from the set of candidate reactants, in particular to a user. This may be performed by displaying the outlier candidates with each subgroup of related additional data on a monitor for the user.

According to another embodiment, the method may also comprise receiving a new plurality of records of updated molecule descriptions, and repeating the step of predicting a reactant of a chemical reaction. Thus, closed loop process may be executed until useful results, i.e. reactants may appear.

In the following, a detailed description of the figures will be given. All instructions in the figures are schematic. Firstly, a block diagram of an embodiment of the inventive computer-implemented method for predicting a reactant of a chemical reaction which results in at least one reaction product, where the at least one reaction product satisfies at least one boundary condition is given. Afterwards, further embodiments, as well as embodiments of the reactant prediction system will be described.

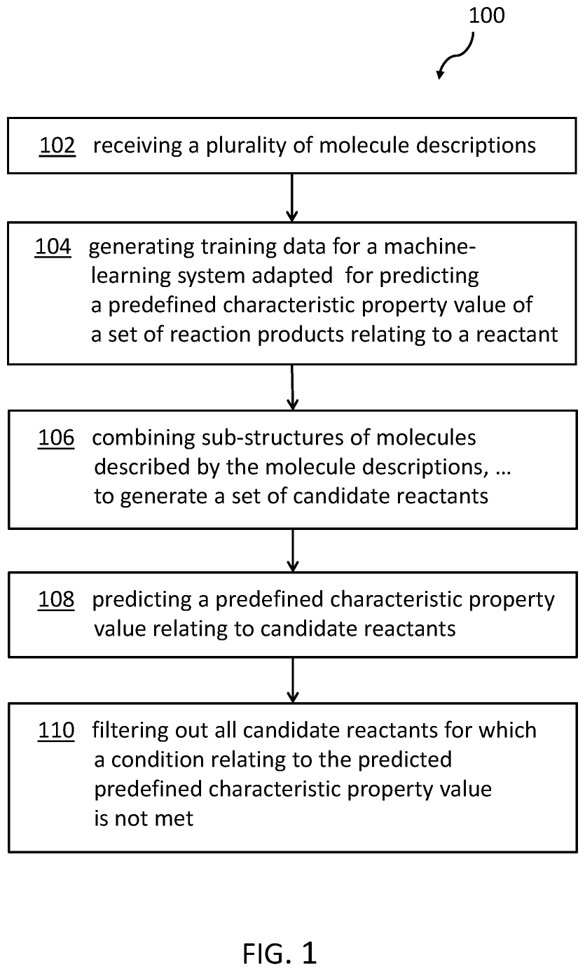

FIG. 1 shows a flow diagram of a computer-implemented method 100 for predicting a reactant of a chemical reaction—in particular, described by a chemical equation—which results in at least one reaction product, where the at least one reaction product satisfies at least one boundary condition. The method 100 comprises receiving, 102—e.g., from a library of molecules—a plurality of records of molecule descriptions. Each record can comprise a codified description of a molecule, e.g., in SMILES notification—and related at least one characteristic property value—e.g., chemical and/or physical property value—of the molecule. The received plurality of records of molecule descriptions can be revised during a subsequent execution of the method, e.g., if the first run did not result in desired products and reactants.

The method 100 also comprises generating, 104, using the plurality of records of molecule descriptions, training data for a machine-learning system adapted for predicting a predefined characteristic property value—the highest boiling point temperature value—of a set of reaction products relating to a reactant, where the reactant R and the set of reaction products relate to each other according to a chemical equation R “reacts to” set of reaction products.

Moreover the method 100 comprises combining, 106, for received ones of the molecule descriptions, sub-structures of the molecules described by the molecule descriptions using chemical rules—in particular by using heuristically known rules—to generate a set of potentially new candidate reactants.

Then, the method 100 comprises predicting, 108, a predefined characteristic property value—in particular, for the reaction products, e.g. boiling point temperature and/or activation energy—relating to each of the set of candidate reactants, whereby the candidate reactants are separately used as input for the trained machine-learning system.

For this the machine-learning system is used in a trained form, where the machine-learning system has been trained using the training data.

Finally, the method comprises, 110, filtering out all candidate reactants for which a condition relating to the predicted predefined characteristic property value is not met. This would be done in such a way that the boundary condition is satisfied.

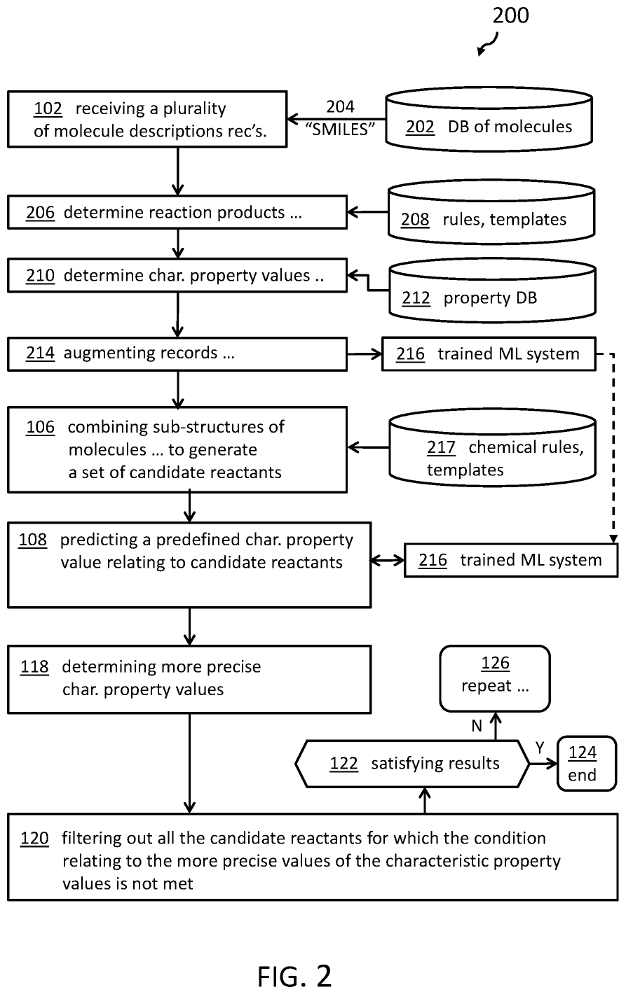

FIG. 2 shows a block diagram 200 of a method and related system components, according to embodiments. It should be noted that alike activities have the same reference numerals as in FIG. 1. Firstly, a plurality of molecule descriptions is received, 102. For this, a database 202 of molecule descriptions may provide the molecule descriptions in a, e.g., SMILES notation. This code may easily be interpreted by a computer system because it is based on a sequence of ASCII characters.

Based on this, for a given (or more) molecule description as reactant, a plurality of potential reaction products is determined. For this, a set of rules, templates 208 and other guarding information may be used. In a next step, characteristic property values of the determined reaction products can be determined, 210. For this, a property value database 212 may be used (e.g., the NIST (US National Institute of Standards and Technology) database or the EPA (US Environmental Protection Agency) TEST (Toxicity Estimation Software Tool) database.

Next, the received records of molecule descriptions are now augmented, 214, with the determined characteristic property values of the determined reaction products for a given reactant (or a plurality thereof).

Using these augmented data records, a machine-learning system is trained to generate the trained machine-learning system, i.e., to generate a specific machine-learning model being adopted to predict characteristic property values of reaction products for a given (e.g., new) reactant. One example of the characteristic property values can be the boiling point temperatures of the reaction products.

As a next step, a combination of sub-structures of the molecules of the received molecule descriptions is combined, 106 (compare FIG. 1), in order to generate a set of candidate reactants. In order to achieve this result, e.g., a database 217 or another information source for chemical rules may be used as well as a trained machine-learning model (underlying the trained ML system) can be used. In one special embodiment, the database 202 and the database 217 may be a combined or the same database.

Using the trained ML system 216, it is possible to predict a value of a predefined characteristic property for a given reactant, 108. It appears to be a natural effect that such prediction is more an estimation of the characteristic property value, e.g., a highest boiling point temperature of reaction products. Thus, a more precise characteristic property value for each of the reaction products can be determined, 118. Existing methods like referring to a database of characteristic property values can be instrumental.

Then, the process comprises filtering out, 120, all the candidate reactants for which the condition relating to the more precise values of the characteristic property values is not met.

If the results are satisfying—compare determination 122—the process ends 124 (case “Y”). If that is not the case—case “N”—the complete process may be repeated, 126. For this, other molecules, or better other molecule descriptions, can be selected from the database of molecules 206. This loop process may be executed until a satisfying set of reaction products—in particular, at least one—has been determined as well as the related reactant.

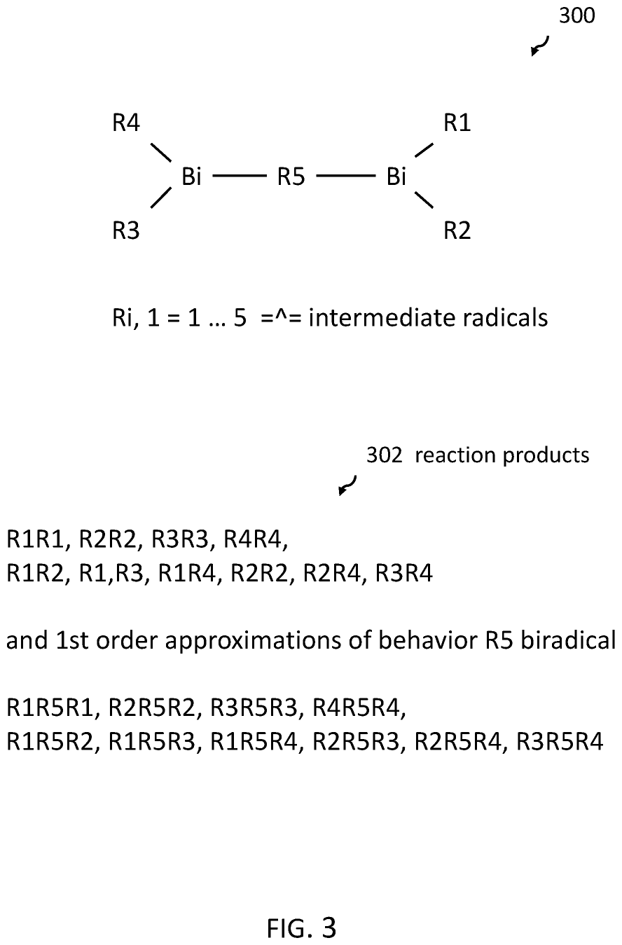

FIG. 3 shows an example of candidate reactant 300 for a boundary condition requiring at least one metal atom, here, Bi (Bismuth) as part of at least one reaction product. Ri, i=1 . . . 5 stands for intermediate (bi-)radicals. If the boundary condition requires as well that the boiling point temperature of all reaction products shall be below a predefined temperature value, e.g., 100° C., then all shown reaction products 302 (R1R1, . . . , R3R5R4) must fulfill this boundary condition.

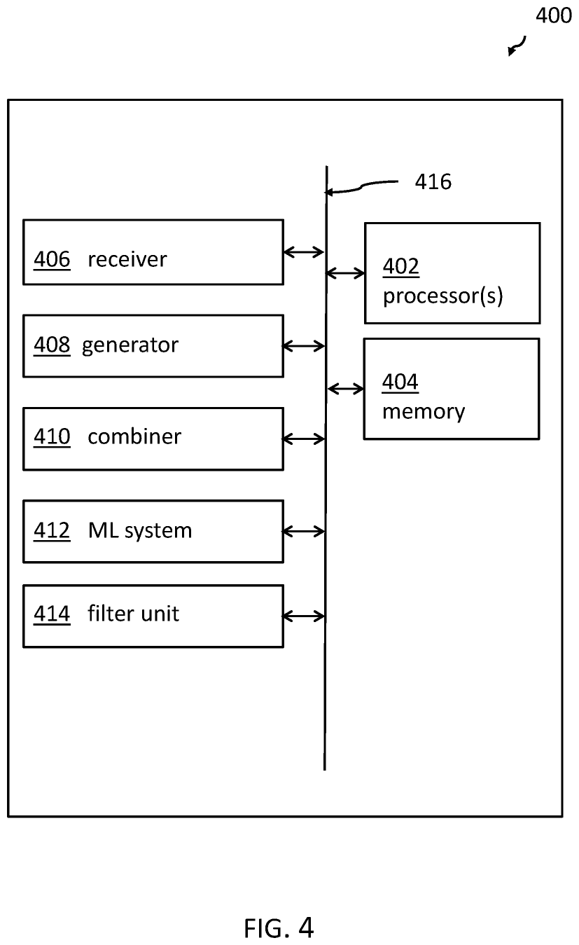

FIG. 4 shows a block diagram of an embodiment of the reactant prediction system 400 for predicting a reactant of a chemical reaction which results in at least one reaction product, wherein the at least one reaction product satisfies at least one boundary condition. The system comprises one or more processors 402 and a memory 404 operatively coupled to the one or more processors 402, where the memory 404 stores program code portions which, when executed by the one or more processors 402, enable the one or more processors to receive—in particular by a receiver 406—a plurality of records of molecule descriptions, wherein each record comprises a codified description of a molecule and related at least one characteristic property value of the molecule, and to generate, using the plurality of records of molecule descriptions by a generator 408, training data for a machine-learning system adapted for predicting a predefined characteristic property value of a set of reaction products relating to a reactant, wherein the reactant R and the set of reaction products relate to each other according to a chemical equation

R→set of reaction products.

The one or more processors 402 are also enabled to combine—in particular, by a combiner 410—sub-structures of molecules described by the molecule descriptions using chemical rules to generate a set of candidate reactants codified as candidate reactant description, and to predict, using the machine-learning system 412 in a trained form, a predefined characteristic property value relating to candidate reactants. Thereby, the candidate reactants are separately used as input for the trained machine-learning system. Additionally, the machine-learning system 412 (compare also FIG. 2, 216) has been trained using the training data.

Finally, the one or more processors 402 are also enabled to filter out—in particular, by a filter unit 414—all candidate reactants for which a condition relating to the predicted predefined characteristic property value is not met.

It shall also be mentioned that all functional units, modules and functional blocks—i.e., the one or more processors 402, the memory 404, the receiver 406, the generator 408, the combiner 410, the ML system 412 and the filter unit 414—may be communicatively coupled to each other for signal or message exchange in a selected 1:1 manner. Alternatively, the functional units, modules and functional blocks can be linked to a system internal bus system 416 for a selective signal or message exchange.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (CPP embodiment or CPP) is a term used in the present disclosure to describe any set of one, or more, storage media (also called mediums) collectively included in a set of one, or more, storage devices that collectively include machine readable code corresponding to instructions and/or data for performing computer operations specified in a given CPP claim. A storage device is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 500 contains an example of an environment for the execution of at least some of the computer code involved in performing the inventive methods, such as the reactant predicting code 550. In addition to block 550, computing environment 500 includes, for example, computer 501, wide area network (WAN) 502, end user device (EUD) 503, remote server 504, public cloud 505, and private cloud 506. In this embodiment, computer 501 includes processor set 510 (including processing circuitry 520 and cache 521), communication fabric 511, volatile memory 512, persistent storage 513 (including operating system 522 and block 550, as identified above), peripheral device set 514 (including user interface (UI), device set 523, storage 524, and Internet of Things (IoT) sensor set 525), and network module 515. Remote server 504 includes remote database 530. Public cloud 505 includes gateway 540, cloud orchestration module 541, host physical machine set 542, virtual machine set 543, and container set 544.

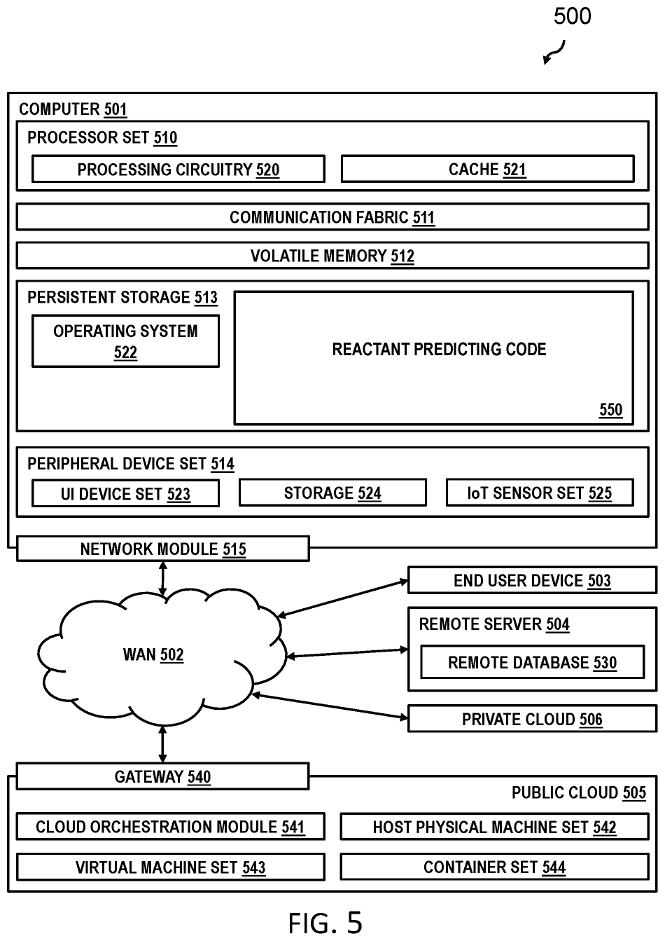

COMPUTER 501 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 530. As is well understood in the art of computer technology, and depending upon the technology, performance of a computer-implemented method may be distributed among multiple computers and/or between multiple locations. On the other hand, in this presentation of computing environment 500, detailed discussion is focused on a single computer, specifically computer 501, to keep the presentation as simple as possible. Computer 501 may be located in a cloud, even though it is not shown in a cloud in FIG. 5. On the other hand, computer 501 is not required to be in a cloud except to any extent as may be affirmatively indicated.

PROCESSOR SET 510 includes one, or more, computer processors of any type now known or to be developed in the future. Processing circuitry 520 may be distributed over multiple packages, for example, multiple, coordinated integrated circuit chips. Processing circuitry 520 may implement multiple processor threads and/or multiple processor cores. Cache 521 is memory that is located in the processor chip package(s) and is typically used for data or code that should be available for rapid access by the threads or cores running on processor set 510. Cache memories are typically organized into multiple levels depending upon relative proximity to the processing circuitry. Alternatively, some, or all, of the cache for the processor set may be located ‘off chip’. In some computing environments, processor set 510 may be designed for working with qubits and performing quantum computing.

Computer readable program instructions are typically loaded onto computer 501 to cause a series of operational steps to be performed by processor set 510 of computer 501 and thereby effect a computer-implemented method, such that the instructions thus executed will instantiate the methods specified in flowcharts and/or narrative descriptions of computer-implemented methods included in this document (collectively referred to as ‘the inventive methods’). These computer readable program instructions are stored in various types of computer readable storage media, such as cache 521 and the other storage media discussed below: The program instructions, and associated data, are accessed by processor set 510 to control and direct performance of the inventive methods. In computing environment 500, at least some of the instructions for performing the inventive methods may be stored in block 550 in persistent storage 513.

COMMUNICATION FABRIC 511 is the signal conduction paths that allow the various components of computer 501 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 512 is any type of volatile memory now known or to be developed in the future. Examples include dynamic type random access memory (RAM) or static type RAM. Typically, the volatile memory is characterized by random access, but this is not required unless affirmatively indicated. In computer 501, the volatile memory 512 is located in a single package and is internal to computer 501, but, alternatively or additionally, the volatile memory may be distributed over multiple packages and/or located externally with respect to computer 501.

PERSISTENT STORAGE 513 is any form of non-volatile storage for computers that is now known or to be developed in the future. The non-volatility of this storage means that the stored data is maintained regardless of whether power is being supplied to computer 501 and/or directly to persistent storage 513. Persistent storage 513 may be a read only memory (ROM), but typically at least a portion of the persistent storage allows writing of data, deletion of data and re-writing of data. Some familiar forms of persistent storage include magnetic disks and solid state storage devices. Operating system 522 may take several forms, such as various known proprietary operating systems or open source Portable Operating System Interface type operating systems that employ a kernel. The code included in block 550 typically includes at least some of the computer code involved in performing the inventive methods.

PERIPHERAL DEVICE SET 514 includes the set of peripheral devices of computer 501. Data communication connections between the peripheral devices and the other components of computer 501 may be implemented in various ways, such as Bluetooth connections, Near-Field Communication (NFC) connections, connections made by cables (such as universal serial bus (USB) type cables), insertion type connections (e.g., secure digital (SD) card), connections made though local area communication networks and even connections made through wide area networks such as the internet. In various embodiments, UI device set 523 may include components such as a display screen, speaker, microphone, wearable devices (such as goggles and smart watches), key board, mouse, printer, touchpad, game controllers, and haptic devices. Storage 524 is external storage, such as an external hard drive, or insertable storage, such as an SD card. Storage 524 may be persistent and/or volatile. In some embodiments, storage 524 may take the form of a quantum computing storage device for storing data in the form of qubits. In embodiments where computer 501 is required to have a large amount of storage (for example, where computer 501 locally stores and manages a large database) then this storage may be provided by peripheral storage devices designed for storing very large amounts of data, such as a storage area network (SAN) that is shared by multiple, geographically distributed computers. IoT sensor set 525 is made up of sensors that can be used in Internet of Things applications. For example, one sensor may be a thermometer and another sensor may be a motion detector.

NETWORK MODULE 515 is the collection of computer software, hardware, and firmware that allows computer 501 to communicate with other computers through WAN 502. Network module 515 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 515 are performed on the same physical hardware device. In other embodiments (e.g., embodiments that utilize software-defined networking (SDN)), the control functions and the forwarding functions of network module 515 are performed on physically separate devices, such that the control functions manage several different network hardware devices. Computer readable program instructions for performing the inventive methods can typically be downloaded to computer 501 from an external computer or external storage device through a network adapter card or network interface included in network module 515.

WAN 502 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 503 is any computer system that is used and controlled by an end user (for example, a customer of an enterprise that operates computer 501), and may take any of the forms discussed above in connection with computer 501. EUD 503 typically receives helpful and useful data from the operations of computer 501. For example, in a hypothetical case where computer 501 is designed to provide a recommendation to an end user, this recommendation would typically be communicated from network module 515 of computer 501 through WAN 502 to EUD 503. In this way, EUD 503 can display, or otherwise present, the recommendation to an end user. In some embodiments, EUD 503 may be a client device, such as thin client, heavy client, mainframe computer, desktop computer and so on.

REMOTE SERVER 504 is any computer system that serves at least some data and/or functionality to computer 501. Remote server 504 may be controlled and used by the same entity that operates computer 501. Remote server 504 represents the machine(s) that collect and store helpful and useful data for use by other computers, such as computer 501. For example, in a hypothetical case where computer 501 is designed and programmed to provide a recommendation based on historical data, then this historical data may be provided to computer 501 from remote database 530) of remote server 504.

PUBLIC CLOUD 505 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 505 is performed by the computer hardware and/or software of cloud orchestration module 541. The computing resources provided by public cloud 505 are typically implemented by virtual computing environments that run on various computers making up the computers of host physical machine set 542, which is the universe of physical computers in and/or available to public cloud 505. The virtual computing environments (VCEs) typically take the form of virtual machines from virtual machine set 543 and/or containers from container set 544. It is understood that these VCEs may be stored as images and may be transferred among and between the various physical machine hosts, either as images or after instantiation of the VCE. Cloud orchestration module 541 manages the transfer and storage of images, deploys new instantiations of VCEs and manages active instantiations of VCE deployments. Gateway 540 is the collection of computer software, hardware, and firmware that allows public cloud 505 to communicate through WAN 502.

Some further explanation of virtualized computing environments (VCEs) will now be provided. VCEs can be stored as ‘images’. A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 506 is similar to public cloud 505, except that the computing resources are only available for use by a single enterprise. While private cloud 506 is depicted as being in communication with WAN 502, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 505 and private cloud 506 are both part of a larger hybrid cloud.

It should also be mentioned that the reactant prediction system 40 for predicting a reactant of a chemical reaction which results in at least one reaction product, wherein the at least one reaction product satisfies at least one boundary condition can be an operational sub-system of the computer 501 and may be attached to a computer-internal bus system.

The terminology used herein is for the purpose of describing particular embodiments only and is not intended to limit the invention. As used herein, the singular forms a, an and the are intended to include the plural forms as well, unless the context clearly indicates otherwise. It will further be understood that the terms comprises and/or comprising, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or steps plus function elements in the claims below are intended to include any structure, material, or act for performing the function in combination with other claimed elements, as specifically claimed. The description of the present invention has been presented for purposes of illustration and description, but is not intended to be exhaustive or limited to the invention in the form disclosed. Many modifications and variations will be apparent to those of ordinary skills in the art without departing from the scope and spirit of the invention. The embodiments are chosen and described in order to best explain the principles of the invention and the practical application, and to enable others of ordinary skills in the art to understand the invention for various embodiments with various modifications, as are suited to the particular use contemplated.

Embodiments of the present disclosure may be summarized by the following clauses:
1. A computer-implemented method for predicting a reactant of a chemical reaction which results in at least one reaction product, wherein the at least one reaction product satisfies at least one boundary condition, the method comprising
   receiving a plurality of records of molecule descriptions, wherein each record comprises a codified description of a molecule and related at least one characteristic property value of the molecule,
   generating, using the plurality of records of molecule descriptions, training data for a machine-learning system adapted for predicting a predefined characteristic property value of a set of reaction products relating to a reactant, wherein the reactant R and the set of reaction products relate to each other according to a chemical equation

R→set of reaction products,
combining, for received records of the molecule descriptions, sub-structures of molecules described by the molecule descriptions using chemical rules to generate a set of candidate reactants,
predicting, using the machine-learning system in a trained form, a predefined characteristic property value relating to candidate reactants, whereby the candidate reactants are separately used as input for the trained machine-learning system,
wherein the machine-learning system has been trained using the training data, and
   filtering out all candidate reactants for which a condition relating to the predicted predefined characteristic property value is not met.
2. The method according to clause 1, wherein the predefined characteristic property value is at least one selected out of the group comprising a highest boiling point temperature of the at least one of the reaction product of a set of reaction products and an activation energy required to facilitate a chemical reaction according to

R→set of reaction products, wherein R is a reactant.
3. The method according to clause 1 or 2, wherein the generating the training data comprises
   determining a set of reaction products for each of the records of molecule descriptions, whereby molecules relating to the molecule descriptions are used as reactants,
   determining a characteristic property value for each reaction product in each set of reaction products being determined for each of the plurality of molecules used as reactants, and
   augmenting each record of molecule descriptions with a predefined value of the characteristic property values of the set of the determined reaction products which is related to the corresponding record of the molecule descriptions.
4. The method according to any of the preceding claims, wherein the filtering out comprises also the
   determining more precise values of the characteristic property values for each reaction product relating to the set of generated candidate reactants, and
   filtering out all the candidate reactants for which the condition relating to the more precise values of the characteristic property values is not met.
5. The method according any of the preceding claims, wherein the reactant R comprises at least two reactants and wherein the reaction products comprise only one reaction product.
6. The method according to any of the preceding claims, wherein the boundary condition or an environmental condition for the chemical reaction is an exposure to a radiation source.
7. The method according to clause 6, wherein the radiation source is an extreme ultraviolet (EUV) radiation source.
8. The method according to any of the preceding claims, wherein the at least one characteristic property value in the plurality of records of molecule descriptions comprises an activation energy for a molecule described by the related molecule description of the record.
9. The method according to any of the preceding claims, wherein the generation the set of candidate reactants also comprises
   identifying outliers in the set of candidate reactants regarding their chemical properties.
10. The method according to clause 9, also comprising
   generating an alert signal and presenting the identified outliers from the set of candidate reactants.
11. The method according to clause 9, also comprising
   receiving a new plurality of records of updated molecule descriptions, and
   repeating the predicting a reactant of a chemical reaction.
12. A reactant prediction system for predicting a reactant of a chemical reaction which results in at least one reaction product, wherein the at least one reaction product satisfies at least one boundary condition, the system comprising
   one or more processors and a memory operatively coupled to the one or more processors, wherein the memory stores program code portions which, when executed by the one or more processors, enable the one or more processors to
   receive a plurality of records of molecule descriptions, wherein each record comprises a codified description of a molecule and related at least one characteristic property value of the molecule,
   generate, using the plurality of records of molecule descriptions, training data for a machine-learning system adapted for predicting a predefined characteristic property value of a set of reaction products relating to a reactant, wherein the reactant R and the set of reaction products relate to each other according to a chemical equation

R→set of reaction products,
combine, for received records of the molecule descriptions, sub-structures of molecules described by the molecule descriptions using chemical rules to generate a set of candidate reactants codified as candidate reactant description,
predict, using the machine-learning system in a trained form, a predefined characteristic property value relating to candidate reactants, whereby the candidate reactants are separately used as input for the trained machine-learning system,
wherein the machine-learning system has been trained using the training data, and
   filter out all candidate reactants for which a condition relating to the predicted predefined characteristic property value is not met.
13. The system according to clause 12, wherein the predefined characteristic property value is at least one selected out of the group comprising a highest boiling point temperature of the at least one of the reaction product of a set of reaction products and an activation energy required to facilitate a chemical reaction according to

R→set of reaction products, wherein
R is a reactant.
14. The system according to clause 12 or 13, wherein the one or more processors are, during the generating the training data, also enabled to
   determine a set of reaction products for each of the records of molecule descriptions, whereby molecules relating to the molecule descriptions are used as reactants,
   determine a characteristic property value for each reaction product in each set of reaction products being determined for each of the plurality of molecules used as reactants, and
   augment each record of molecule descriptions with a predefined value of the characteristic property values of the set of the determined reaction products which is related to the corresponding record of the molecule descriptions.
15. The system according to any of the clauses 12 to 14, wherein the one or more processors are, during the filtering out, also enabled to
   determine more precise values of the characteristic property values for each reaction product relating to the set of generated candidate reactants, and
   filter out all the candidate reactants for which the condition relating to the more precise values of the characteristic property values is not met.
16. The system according to any of the clauses 12 to 15, wherein the reactant R comprises at least two reactants and wherein the reaction products comprise only one reaction product.
17. The system according to any of the clauses 12 to 16, wherein the at least one characteristic property value in the plurality of records of molecule descriptions comprises an activation energy for a molecule described by the related molecule description of the record.
18. The system according to any of the clauses 12 to 17, wherein the one or more processors are, during the generation the set of candidate reactants, also enabled to
   identify outliers in the set of candidate reactants regarding their chemical properties.
19. The system according to clause 18, the one or more processors are also enabled to
   generate an alert signal and presenting the identified outliers from the set of candidate reactants.
20. A computer program product for predicting a reactant of a chemical reaction which results in at least one reaction product, wherein the at least one reaction product satisfies at least one boundary condition, the computer program product comprising a computer readable storage medium having program instructions embodied therewith, the program instructions being executable by one or more computing systems or controllers to cause the one or more computing systems to
   receive a plurality of records of molecule descriptions, wherein each record comprises a codified description of a molecule and related at least one characteristic property value of the molecule,
   generate, using the plurality of records of molecule descriptions, training data for a machine-learning system adapted for predicting a predefined characteristic property value of a set of reaction products relating to a reactant, wherein the reactant R and the set of reaction products relate to each other according to a chemical equation

R→set of reaction products,
combine, for received records of the molecule descriptions, sub-structures of molecules described by the molecule descriptions using chemical rules to generate a set of candidate reactants codified as candidate reactant description,
predict, using the machine-learning system in a trained form, a predefined characteristic property value relating to candidate reactants, whereby the candidate reactants are separately used as input for the trained machine-learning system, wherein the machine-learning system has been trained using the training data, and
filter out all candidate reactants for which a condition relating to the predicted predefined characteristic property value is not met.

What is claimed is:

1. A computer-implemented method for predicting a reactant of a chemical reaction which results in at least one reaction product, wherein the at least one reaction product satisfies at least one boundary condition, the method comprising:
receiving a plurality of records of molecule descriptions, wherein each record comprises a codified description of a molecule and related at least one characteristic property value of the molecule;
generating, using the plurality of records of molecule descriptions, training data for a machine-learning system adapted for predicting a predefined characteristic property value of a set of reaction products relating to a reactant, wherein the reactant R and the set of reaction products relate to each other according to a chemical equation
R→set of reaction products:
combining, for received records of the molecule descriptions, sub-structures of molecules described by the molecule descriptions using chemical rules to generate a set of candidate reactants;
predicting, using the machine-learning system in a trained form, a predefined characteristic property value relating to candidate reactants, whereby the candidate reactants are separately used as input for the trained machine-learning system, wherein the machine-learning system has been trained using the training data; and
filtering out all candidate reactants for which a condition relating to the predicted predefined characteristic property value is not met.

2. The method of claim 1, wherein the predefined characteristic property value is at least one selected out of the group comprising a highest boiling point temperature of the at least one reaction product of a set of reaction products and an activation energy required to facilitate a chemical reaction according to
R→set of reaction products, wherein R is a reactant.

3. The method of claim 1, wherein the generating the training data comprises:
determining a set of reaction products for each of the records of molecule descriptions, whereby molecules relating to the molecule descriptions are used as reactants;
determining a characteristic property value for each reaction product in each set of reaction products being determined for each of the plurality of molecules used as reactants; and
augmenting each record of molecule descriptions with a predefined value of the characteristic property values of the set of the determined reaction products which is related to the corresponding record of the molecule descriptions.

4. The method of claim 1, wherein the filtering out comprises determining more precise values of the characteristic property values for each reaction product relating to the set of generated candidate reactants, and filtering out all the candidate reactants for which the condition relating to the more precise values of the characteristic property values is not met.

5. The method of claim 1, wherein the reactant R comprises at least two reactants and wherein the reaction products comprise only one reaction product.

6. The method of claim 1, wherein the boundary condition or an environmental condition for the chemical reaction is an exposure to a radiation source.

7. The method of claim 6, wherein the radiation source is an extreme ultraviolet (EUV) radiation source.

8. The method of claim 1, wherein the at least one characteristic property value in the plurality of records of molecule descriptions comprises an activation energy for a molecule described by the related molecule description of the record.

9. The method of claim 1, wherein the generation the set of candidate reactants further comprises identifying outliers in the set of candidate reactants regarding their chemical properties.

10. The method of claim 9, further comprising generating an alert signal and presenting the identified outliers from the set of candidate reactants.

11. The method of claim 9, further comprising:
receiving a new plurality of records of updated molecule descriptions; and
repeating the predicting a reactant of a chemical reaction.

12. A reactant prediction system for predicting a reactant of a chemical reaction which results in at least one reaction product, wherein the at least one reaction product satisfies at least one boundary condition, the system comprising:
one or more processors and a memory operatively coupled to the one or more processors, wherein the memory stores program code portions which, when executed by the one or more processors, enable the one or more processors to:
receive a plurality of records of molecule descriptions, wherein each record comprises a codified description of a molecule and related at least one characteristic property value of the molecule;
generate, using the plurality of records of molecule descriptions, training data for a machine-learning system adapted for predicting a predefined characteristic property value of a set of reaction products relating to a reactant, wherein the reactant R and the set of reaction products relate to each other according to a chemical equation
R→set of reaction products:
combine, for received records of the molecule descriptions, sub-structures of molecules described by the molecule descriptions using chemical rules to generate a set of candidate reactants codified as candidate reactant description:
predict, using the machine-learning system in a trained form, a predefined characteristic property value relating to candidate reactants, whereby the candidate reactants are separately used as input for the trained machine-learning system, wherein the machine-learning system has been trained using the training data, and
filter out all candidate reactants for which a condition relating to the predicted predefined characteristic property value is not met.

13. The system of claim 12, wherein the predefined characteristic property value is at least one selected out of the group comprising a highest boiling point temperature of the at least one of the reaction product of a set of reaction products and an activation energy required to facilitate a chemical reaction according to
R→set of reaction products, wherein R is a reactant.

14. The system of claim 12, wherein the one or more processors are, during the generating the training data, further enabled to:
determine a set of reaction products for each of the records of molecule descriptions, whereby molecules relating to the molecule descriptions are used as reactants;
determine a characteristic property value for each reaction product in each set of reaction products being determined for each of the plurality of molecules used as reactants; and
augment each record of molecule descriptions with a predefined value of the characteristic property values of the set of the determined reaction products which is related to the corresponding record of the molecule descriptions.

15. The system of claim 12, wherein the one or more processors are, during the filtering out, further enabled to:
determine more precise values of the characteristic property values for each reaction product relating to the set of generated candidate reactants; and
filter out all the candidate reactants for which the condition relating to the more precise values of the characteristic property values is not met.

16. The system of claim 12, wherein the reactant R comprises at least two reactants and wherein the reaction products comprise only one reaction product.

17. The system of claim 12, wherein the at least one characteristic property value in the plurality of records of molecule descriptions comprises an activation energy for a molecule described by the related molecule description of the record.

18. The system of claim 12, wherein the one or more processors are, during the generation the set of candidate reactants, further enabled to identify outliers in the set of candidate reactants regarding their chemical properties.

19. The system of claim 18, the one or more processors are further enabled to generate an alert signal and presenting the identified outliers from the set of candidate reactants.

20. A computer program product for predicting a reactant of a chemical reaction which results in at least one reaction product, wherein the at least one reaction product satisfies at least one boundary condition, the computer program product comprising a computer readable storage medium having program instructions embodied therewith, the program instructions being executable by one or more processors to cause the one or more processors to:
receive a plurality of records of molecule descriptions, wherein each record comprises a codified description of a molecule and related at least one characteristic property value of the molecule;
generate, using the plurality of records of molecule descriptions, training data for a machine-learning system adapted for predicting a predefined characteristic property value of a set of reaction products relating to a reactant, wherein the reactant R and the set of reaction products relate to each other according to a chemical equation
R→set of reaction products:
combine, for received records of the molecule descriptions, sub-structures of molecules described by the molecule descriptions using chemical rules to generate a set of candidate reactants codified as candidate reactant description;
predict, using the machine-learning system in a trained form, a predefined characteristic property value relating to candidate reactants, whereby the candidate reactants are separately used as input for the trained machine-learning system, wherein the machine-learning system has been trained using the training data; and
filter out all candidate reactants for which a condition relating to the predicted predefined characteristic property value is not met.
